(19) United States
(12) Patent Application Publication
(10) Pub. No.: US 2019/0255287 A1
(43) Pub. Date: Aug. 22, 2019

(54) ADJUSTABLE CATHETER STRAIGHTENER

(71) Applicant: St. Jude Medical, Cardiology Division, Inc., St. Paul, MN (US)

(72) Inventors: Xuan Yen Khieu, Maple Grove, MN (US); John Hong, Prior Lake, MN (US)

(21) Appl. No.: 16/344,330

(22) PCT Filed: Oct. 23, 2017
(86) PCT No.: PCT/US2017/057845

§ 371 Date: Apr. 23, 2019

Related U.S. Application Data
(60) Provisional application No. 62/411,979, filed on Oct. 24, 2016.

(51) Int. Cl. A61M 25/01; A61M 25/06; A61M 29/02

(52) U.S. Cl. CPC A61M 25/0102; A61M 2025/0175; A61M 29/02; A61M 25/0662

(57) ABSTRACT

Various embodiments of the present disclosure can provide an adjustable catheter straightener (102). The adjustable catheter straightener can include a first elongate shaft (104) extending along a shaft longitudinal axis and can include a first shaft proximal end (106) and a first shaft distal end (108). A first shaft inner wall (110) of the first elongate shaft can define a first shaft lumen (112) extending therethrough. A second elongate shaft (114) is disposed within the first shaft lumen extending along the shaft longitudinal axis and comprises a second shaft proximal end and a second shaft distal end. A second shaft inner wall of the second elongate shaft defines a second shaft lumen extending therethrough. The first shaft lumen and the second shaft lumen form a continuous lumen and the first elongate shaft and the second elongate shaft are configured to move longitudinally with respect to one another.

### BACKGROUND

### a. Field

The instant disclosure relates generally to adjustable catheter straighteners.

### b. Background

Medical devices, such as introducer catheters, balloon catheters, dilatation catheters, and other similar devices can be used in a variety of diagnostic, therapeutic, and/or mapping and ablation procedures to diagnose and/or correct various cardiac conditions. Oftentimes, an introducer catheter or sheath can be used to guide a circular catheter, a balloon catheter, or other type of catheter into the body of a patient. The introducer catheter can include a hemostasis valve at its proximal end for preventing blood loss when the introducer is placed in a venous and/or arterial system. Since less blood is lost with a hemostasis valve, the need for blood transfusions may be reduced.

A catheter straightener may be used to facilitate insertion of a catheter into a hemostasis valve of an introducer catheter. Some catheter straighteners can comprise a solid, polymeric tube placed over a portion of the catheter, such that the catheter is housed in the catheter straightener. The distal end of the catheter straightener can be inserted into the proximal end of the hemostasis valve of the introducer catheter and the catheter can be distally advanced from the catheter straightener into the introducer catheter. The catheter straightener can aid in the alignment of the catheter and in some cases can help open the hemostasis valve to allow the catheter to be inserted into the introducer catheter and eventually into the body of the patient. The catheter straightener should have a sufficient column strength to avoid buckling as the catheter straightener is advanced through a hemostasis valve. However, portions of the catheter straightener should have a flexibility that prevents damage to a medical device (e.g., catheter) being inserted through the catheter straightener.

### BRIEF SUMMARY

Various embodiments of the present disclosure can provide an adjustable catheter straightener. The adjustable catheter straightener can include a first elongate shaft extending along a shaft longitudinal axis and can include a first shaft proximal end and a first shaft distal end. A first shaft inner wall of the first elongate shaft can define a first shaft lumen extending therethrough. A second elongate shaft can be disposed within the first shaft lumen extending along the shaft longitudinal axis and can comprise a second shaft proximal end and a second shaft distal end. A second shaft inner wall of the second elongate shaft can define a second shaft lumen extending therethrough. The first shaft lumen and the second shaft lumen can form a continuous lumen and the first elongate shaft and the second elongate shaft can be configured to move longitudinally with respect to one another.

Various embodiments of the present disclosure can provide an adjustable catheter straightener. The adjustable catheter straightener can include a first elongate shaft that extends along a shaft longitudinal axis and can comprise a first shaft proximal end and a first shaft distal end. A first shaft inner wall of the first elongate shaft can define a first shaft lumen extending therethrough. A second elongate shaft can be disposed within the first shaft lumen and can extend along the shaft longitudinal axis and can comprise a second shaft proximal end and a second shaft distal end. A second shaft inner wall of the second elongate shaft can define a second shaft lumen extending therethrough. A flexible tip can be disposed at the second shaft distal end. A tip inner wall of the flexible tip can define a tip lumen, wherein the first shaft lumen, the second shaft lumen, and the tip lumen form a continuous lumen.

Various embodiments of the present disclosure can provide an adjustable catheter straightener. The adjustable catheter straightener can include a first elongate shaft that extends along a shaft longitudinal axis and can include a first shaft proximal end and a first shaft distal end. A first shaft inner wall of the first elongate shaft can define a first shaft lumen extending therethrough. A second elongate shaft can be disposed within the first shaft lumen and can extend along the shaft longitudinal axis and can include a second shaft proximal end and a second shaft distal end. A second shaft inner wall of the second elongate shaft can define a second shaft lumen extending therethrough. A flexible tip can be disposed at the second shaft proximal end. A tip inner wall of the flexible tip can define a tip lumen. The first shaft lumen, the second shaft lumen, and the tip lumen can form a continuous lumen. A locking device can be configured to prevent longitudinal movement between the first elongate shaft and the second elongate shaft.

### BRIEF DESCRIPTION OF THE DRAWINGS

FIG. 1 is a partial cross-sectional side view of a catheter straightener, in accordance with embodiments of the present disclosure.

FIG. 2A is a schematic and isometric side, top, and proximal end view of an adjustable catheter straightener with a collet, in accordance with embodiments of the present disclosure.

FIG. 2B depicts a cross-sectional side view of the collet depicted in FIG. 2A along line 2B-2B, in accordance with embodiments of the present disclosure.

FIG. 2C is a schematic and isometric side, top, and proximal end view of the adjustable catheter straightener in FIG. 2A in an extended state, in accordance with embodiments of the present disclosure.

FIG. 3A is a side, top, and proximal end view of the adjustable catheter straightener in an extended state, in accordance with embodiments of the present disclosure.

FIG. 3B is a cross-sectional view of the collet and the first shaft distal end in FIG. 3A, in accordance with embodiments of the present disclosure.

FIG. 4 is a schematic and isometric side, top, and proximal end view of an adjustable catheter straightener that includes a static locking device, in accordance with embodiments of the present disclosure.

FIG. 5A depicts a cross-sectional side view of an adjustable catheter straightener in a partially extended state with an interface sealed disposed between a first elongate shaft and a second elongate shaft, in accordance with embodiments of the present disclosure.

FIG. 5B depicts a cross-sectional side view of the adjustable catheter straightener in FIG. 5A in a partially retracted state, in accordance with embodiments of the present disclosure.

FIG. 5C is a cross-sectional side, top, and proximal end view of the transition point associated with the interface and the first elongate shaft depicted in FIG. 5A, in accordance with embodiments of the present disclosure.

### DETAILED DESCRIPTION

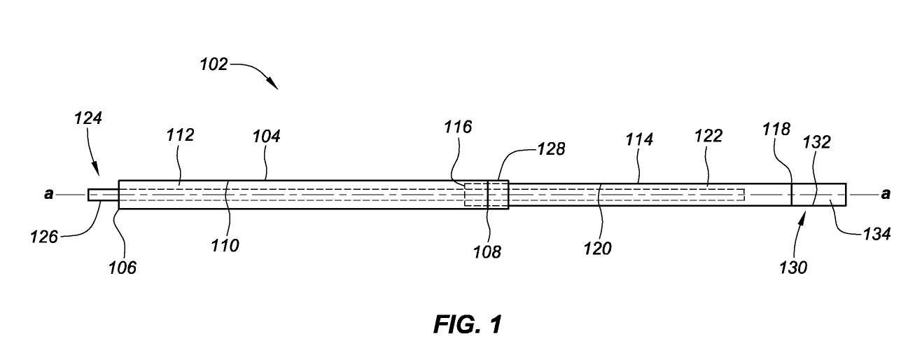

FIG. 1 is a partial cross-sectional side view of a catheter straightener 102, in accordance with embodiments of the present disclosure. The catheter straightener 102 can include a first elongate shaft 104 that extends along a shaft longitudinal axis, defined by line aa. The first elongate shaft 104 can include a first shaft proximal end 106 and a first shaft distal end 108. In some embodiments, the first elongate shaft 104 can include a first shaft inner wall 110 that defines a first shaft lumen 112 that extends therethrough. In some embodiments, the catheter straightener 102 can include a second elongate shaft 114 that is disposed within the first shaft lumen 112 and that extends along the shaft longitudinal axis aa and comprises a second shaft proximal end 116 and a second shaft distal end 118. In some embodiments, a second shaft inner wall 120 of the second elongate shaft 114 can define a second shaft lumen 122 that extends therethrough. In some embodiments, the first shaft lumen 112 and the second shaft lumen 122 can form a continuous lumen that extends from the first shaft proximal end 106 to the second shaft distal end 118.

In some embodiments, the first elongate shaft 104 and the second elongate shaft 114 can be formed from a material that is compatible with blood and other body fluids/tissue. In some embodiments, the first elongate shaft 104 and the second elongate shaft 114 can comprise a biocompatible polymer material in accordance with embodiments of the present disclosure. For example, the first elongate shaft 104 and the second elongate shaft 114 can comprise Nylon (e.g., Nylon-11), high density polyethylene (HDPE), a polyether block amide (Pebax®), etc. The first elongate shaft 104 and the second elongate shaft 114 can be flexible; however, the first elongate shaft 104 and the second elongate shaft 114 can have sufficient rigidity to facilitate insertion of the first elongate shaft 104 and/or the second elongate shaft 114 into a hemostasis valve. In some embodiments, the first elongate shaft 104 and/or the second elongate shaft 114 can be formed from a material that has a durometer in a range from 55D to 72D. However, the elongate shafts can be formed from other materials with a durometer that is less than 55D or greater than 72D. In some embodiments, the first elongate shaft 104 and/or the second elongate shaft 114 can be formed from a braided tubing (e.g., stainless steel braided tubing).

In some embodiments, an outer diameter of the second elongate shaft 114 can be less than an inner diameter of the first elongate shaft 104. In an example, this can provide for a slip fit between the first elongate shaft 104 and the second elongate shaft 114, allowing the second elongate shaft 114 to slide longitudinally into or out of the first shaft lumen 112. For instance, the second elongate shaft 114 can be slid proximally into the first shaft lumen 112 or can be slid distally out of the first shaft lumen 112. This can increase or decrease an overall longitudinal length of the catheter straightener 102, in some embodiments, which can be beneficial when the catheter straightener 102 is used to introduce a catheter into the body of a patient.

In an example, an introducer catheter can be used in conjunction with the catheter straightener 102 to introduce a catheter into the body of a patient. For instance, an introducer catheter can be used to guide a circular catheter, a balloon catheter, or other type of catheter into the body of a patient. In some embodiments, an introducer catheter can include an elongate introducer shaft that extends along a longitudinal axis and defines an introducer lumen that extends therethrough. A distal end of the introducer shaft can be inserted into a venous and/or arterial system, providing access to the venous and/or arterial system via the introducer lumen. A proximal end of the introducer shaft can be connected with a hemostasis valve, in some embodiments. The hemostasis valve can include a casing that surrounds an internal structure of the hemostasis valve. In some embodiments, the internal structure of the hemostasis valve can create a seal between the introducer lumen and a proximal side of the hemostasis valve. The internal structure of the hemostasis valve can generally include a seal formed from a flexible material or another type of seal, such as those discussed in relation to U.S. Pat. No. 8,905,973, which is hereby incorporated by referenced as though fully set forth herein.

When the distal end of the introducer shaft is inserted into the venous and/or arterial system, the internal structure can form a seal, preventing bodily fluid from passing through the introducer lumen and out the proximal side of the hemostasis valve. In some embodiments, the hemostasis valve can allow for a medical device (e.g., catheter, catheter straightener) to be inserted through a proximal end of the hemostasis valve and into the introducer lumen. The internal structure of the hemostasis valve can form a seal around the medical device, preventing bodily fluid from passing out of the proximal end of the hemostasis valve. In an example, the internal structure of the hemostasis valve can deform to create an intimate seal around the medical device. In some embodiments, the medical device can include a catheter straightener 102 and/or a catheter 124.

The catheter 124 can be inserted in a lumen of the catheter straightener 102 formed by the first shaft lumen 112 and the second shaft lumen 122, portions of which that are inserted in the first shaft lumen 112 and the second shaft lumen 122 are depicted in phantom. The catheter 124 can include an elongate catheter shaft 126 that extends along the shaft longitudinal axis aa and can include a proximal end and a distal end, the proximal end of which can be connected to a catheter handle, although not shown. The elongate catheter shaft 126 can comprise a biocompatible polymer material and can include a flexibility that allows it to be threaded through a tortuous venous and/or arterial system. This flexibility and/or design of the distal end of the catheter shaft 126 can make it difficult to insert the catheter shaft 126 through the hemostasis valve. In an example, some force can be required to penetrate the internal structure of the hemostasis valve. However, the catheter shaft 126 may have a flexibility and/or a distal tip design that makes such penetration difficult.

Accordingly, the catheter shaft 126 can be disposed inside of the first shaft lumen 112 and/or the second shaft lumen 122 of the catheter straightener 102, which can generally be more rigid than the catheter shaft 126. The catheter straightener 102 can be beneficial to devices that have unique curved or formed distal designs, such as a circular mapping catheter, and/or devices with flexible distal end portions, for instance. The second shaft distal end 118 and/or flexible tip portion of the catheter straightener 102, further discussed herein, can be inserted partially into or through the internal structure of the hemostasis valve, allowing for the catheter 124 to be advanced into the introducer lumen.

In some embodiments, as the catheter 124 is advanced into the introducer lumen, the catheter 124 can be advanced distally through the catheter straightener 102 into the introducer lumen and ultimately into the patient's vasculature system. However, advancement of the catheter 124 can be limited by the catheter handle attached to the proximal end of the catheter 124. For example, the catheter handle can contact the first shaft proximal end 106. Some approaches have solved this problem by creating a catheter straightener that can be peeled apart. Accordingly, some catheter straighteners can be peeled apart and removed from the catheter 124. However, embodiments of the present disclosure can allow for a longitudinal length of the catheter straightener 102 to be adjusted, thus allowing more room for the catheter 124 and an associated catheter handle to be distally advanced. For example, the longitudinal length of the catheter straightener 102 can be shortened to allow for the catheter 124 and associated catheter handle to be further advanced distally. By shortening the longitudinal length of the catheter straightener 102, a length between the proximal end of the catheter straightener 102 and the hemostasis valve and/or access point into the vasculature system of the patient can be reduced, thus allowing the catheter 124 to be more fully advanced into the vasculature system of the patient.

In some embodiments, the catheter straightener 102 can include a locking device 128 that can be configured to prevent longitudinal movement between the first elongate shaft 104 and the second elongate shaft 114. In some embodiments, the locking device 128 can be disposed at an interface between the first elongate shaft 104 and the second elongate shaft 114. In some embodiments, the locking device 128 can be an adjustable locking device that can apply an adjustable amount of friction between the first elongate shaft 104 and the second elongate shaft 114, between the first elongate shaft 104 and the locking device 128 and/or between the second elongate shaft 114 and the locking device 128.

In some embodiments, the locking device 128 can be a collet that is connected with a distal end of the first elongate shaft 104, as further discussed herein. In some embodiments, the locking device 128 can be a static locking device. In an example, a static locking device can include a frictional fit between the first elongate shaft 104 and the second elongate shaft 114. For instance, the outer diameter of the second elongate shaft 114 can be the same or slightly larger than the inner diameter of the first elongate shaft 104, which can create friction between the first elongate shaft 104 and the second elongate shaft 114, preventing unintentional longitudinal movement between the first elongate shaft 104 and the second elongate shaft 114. However, the amount of friction between the first elongate shaft 104 and the second elongate shaft 114 can be overcome by a user moving the first elongate shaft 104 and the second elongate shaft 114 along the shaft longitudinal axis aa towards one another or away from one another. In some embodiments, a distal inner circumference of the first elongate shaft 104 can be tapered and a proximal outer circumference of the second elongate shaft 114 can be flared. As the first elongate shaft 104 and the second elongate shaft 114 are moved longitudinally apart from one another, the tapered and flared portions can contact one another, causing additional friction (e.g., an interference fit) between the first elongate shaft 104 and the second elongate shaft 114, maintaining a fixed relationship between the first elongate shaft 104 and the second elongate shaft 114. However, as a physician moves the first elongate shaft 104 and the second elongate shaft 114 longitudinally toward one another, the tapered and flared portions no longer contact one another, allowing for the shafts to be slid towards one another and the overall longitudinal length of the catheter straightener 102 to be reduced.

In some embodiments, the second shaft distal end can include a flexible tip 130. The flexible tip 130 can be formed from a material with a flexibility that is greater than the first and/or second elongate shafts, in some embodiments. In some embodiments, the flexible tip 130 can be formed from a material that has a durometer in a range of from 25D to 55D. In some embodiments, the flexible tip 130 can be formed from a material that has a durometer in a range of from 35D to 45D. The flexible tip can be disposed at the second shaft distal end and a tip inner wall 132 of the flexible tip can define a tip lumen 134. In some embodiments, the first shaft lumen, 112, the second shaft lumen 122, and the tip lumen 134 can define a continuous lumen.

The flexible tip 130 can extend along the longitudinal axis aa, distally from the second shaft distal end 118. In some embodiments, the flexible tip 130 can have a length in a range from 0.050 to 0.500. The flexible tip 130 can improve an ease at which the catheter straightener 102 is inserted into the introducer catheter and/or can reduce a damage to the distal end of the catheter straightener 102 and/or the catheter shaft 126 that is inserted through the catheter straightener 102. In some approaches, the distal tip of the catheter straightener 102 may be damaged as a result of not being hard enough. For example, the distal tip of the catheter straightener can be deformed as a result of the distal tip being inserted into the introducer catheter and the distal tip of the catheter straightener not being formed from a material that is hard enough. For instance, some introducer catheters can require a significant amount of force to be exerted on the introducer catheter to cause the introducer to penetrate the hemostasis valve of the introducer catheter. As a result, the distal tip of the introducer can be deformed, in some embodiments.

In other approaches, the distal tip of a catheter being inserted through the catheter straightener can be damaged by the distal tip of the catheter straightener as a result of the distal tip of the catheter straightener being formed from a material that is too hard. For example, when the distal tip of the catheter straightener is formed from a material that is too hard, as the distal tip of the catheter is inserted through the distal tip of the catheter straightener, electrodes or other features disposed on the distal tip of the catheter can be damaged. Embodiments of the present disclosure can provide a solution to this problem through the inclusion of a flexible tip, as discussed herein.

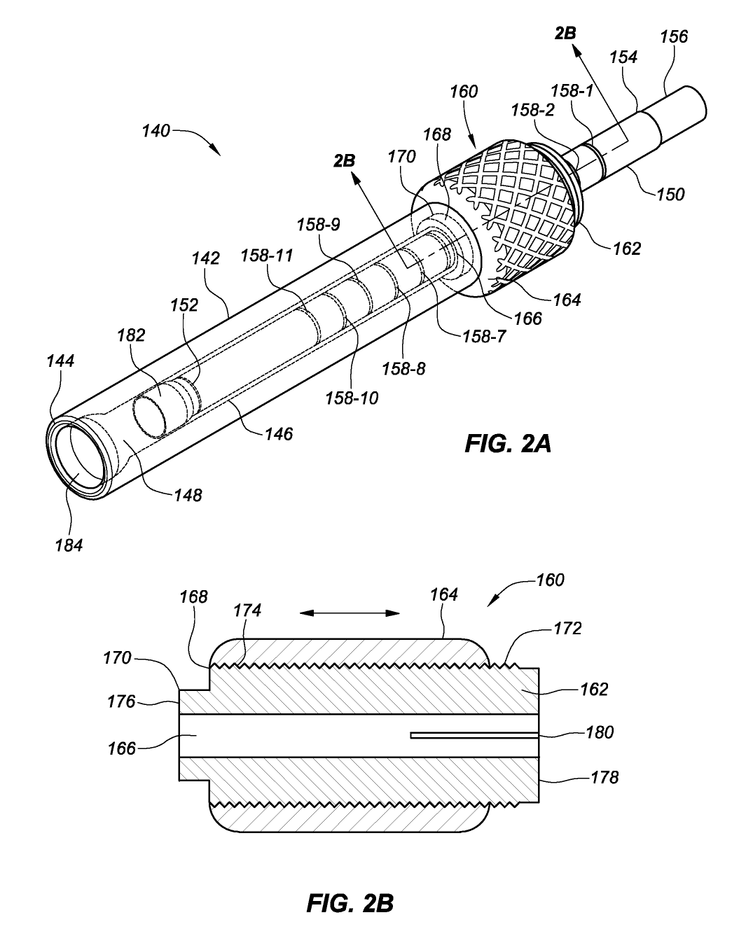

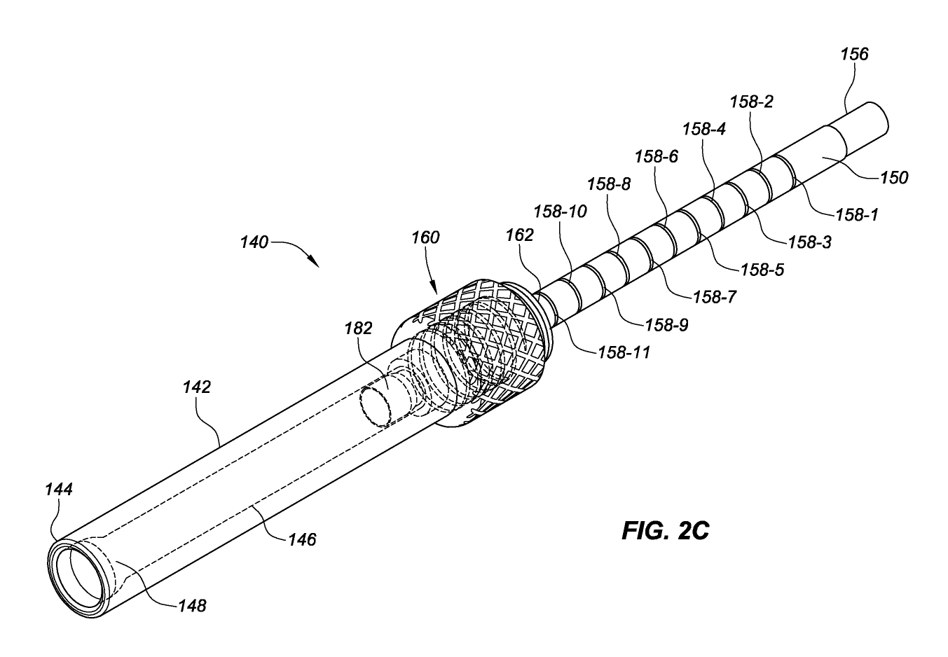

FIG. 2A is a schematic and isometric side, top, and proximal end view of an adjustable catheter straightener 140 with a collet 160, in accordance with embodiments of the present disclosure. The adjustable catheter straightener 140 can include a first elongate shaft 142 that extends along a shaft longitudinal axis. The first elongate shaft 142 can include a first shaft proximal end 144 and a first shaft distal end (not depicted). In some embodiments, the first elongate shaft 142 can include a first shaft inner wall 146 that defines a first shaft lumen 148 that extends therethrough. In some embodiments, the catheter straightener 140 can include a second elongate shaft 150 that is disposed within the first shaft lumen 148 and that extends along the shaft longitudinal axis and comprises a second shaft proximal end 152 and a second shaft distal end 154. In some embodiments, a second shaft inner wall of the second elongate shaft 150 can define a second shaft lumen (not depicted) that extends therethrough. In some embodiments, the first shaft lumen 148 and the second shaft lumen can form a continuous lumen that extends from the first shaft proximal end 144 to the second shaft distal end 154.

In some embodiments, the first elongate shaft 142 can include a proximal flared lumen 184. In an example, the first shaft inner wall 146 can be flared outward toward a proximal end of the first elongate shaft 142, which can result in an increased diameter of the first shaft lumen 148 at the proximal end of the first elongate shaft 142. The increased diameter of the first shaft lumen 148 can allow for a catheter to be more easily inserted into the proximal end of the catheter straightener 140. Additionally, the increased diameter of the first shaft lumen 148 at the proximal end can allow for an easier assembly of the catheter straightener 140. For example, a distal end of the second elongate shaft 150 can be inserted into the proximal end of the first shaft lumen 148 and distally advanced through the first shaft lumen 148. As a seal 182, further discussed below, is distally advanced into the first shaft lumen 148, the seal can gradually be compressed by the first shaft inner wall 146 that defines the proximal flared lumen 184.

As previously discussed in relation to FIG. 1, the distal end of the second elongate shaft 150 can include a flexible tip 156. The flexible tip 156 can define a tip lumen, which together with the first shaft lumen 148 and the second shaft lumen can define a continuous lumen. In some embodiments, the flexible tip 156 can have an outer diameter that is less than, the same as, and/or greater than an outer diameter of the second elongate shaft 150. In some embodiments where an outer diameter of the flexible tip 156 is less than the outer diameter of the second elongate shaft 150, an amount of force required to insert the flexible tip 156 and/the catheter straightener 140 into an introducer catheter can be reduced.

In some embodiments, the second shaft proximal end 152 of the second elongate shaft 150 can include a seal 182. In an example, the seal 182 can be connected to the second shaft proximal end 152, in some embodiments. In some embodiments, the seal 182 can be a hollow cylindrical seal that extends proximally from the second shaft proximal end 152. An outer diameter of the seal 182 can be greater than an outer diameter of the second elongate shaft 150 and can be the same or greater than the inner diameter of the first shaft lumen 148. In an example, in its natural state, seal 182 can have a diameter that is greater than the inner diameter of the first shaft lumen 148. Upon insertion of the seal 182 into the first shaft lumen 148, however, the seal 182 can be compressed by the first shaft inner wall 146, forming a fluid tight seal between the first shaft inner wall 146, flexible tip 156, and the seal 182. In an example, the seal can minimize back bleeding/leaking at the interface between the first elongate shaft 142 and the second elongate shaft 150. In some embodiments, the first elongate shaft 142, the seal 182, and the second elongate shaft 150 can form a continuous lumen that extends from the proximal end of the first elongate shaft 142 to the distal end of the flexible tip 156. A catheter can be inserted through the continuous lumen and into an introducer catheter, as discussed herein.

As depicted, the seal 182 extends proximally from a proximal end of the second elongate shaft 150. However, in some embodiments, a seal can circumferentially extend around an outer surface of the second elongate shaft 150. In an example, a seal can circumferentially extend around the outer surface of a proximal end portion of the second elongate shaft 150. An outer diameter of the seal can be the same or larger than the inner diameter of the first shaft lumen 148 defined by the first shaft inner wall 146. In some embodiments, the seal can be an O-ring disposed around the proximal end portion of the second elongate shaft 150.

In some embodiments, the second elongate shaft 150 can include gradation indicators 158-1, 158-2, . . . , 158-11. Hereinafter, the gradation indicators 158-1, 158-2, . . . , 158-11 are referred to in the plural as gradation indicators 158. Gradation indicators 158-3, 158-4, 158-5, 158-6 are hidden from view, but are depicted in FIG. 2B. In some embodiments, the gradation indicators 158 can indicate a length at which the second elongate shaft 150 has been extended from the first elongate shaft 142. In an example, a length between each gradation indicator 158 can be a defined value. Accordingly, a physician can reference the gradation indicators 158 to determine how far to protract and/or retract the second elongate shaft 150 from the first elongate shaft 142.

In some embodiments, the gradation indicators 158 can circumferentially extend around the second elongate shaft 150 and can be visually distinct from adjacent portions of the second elongate shaft 150. In an example, the gradation indicators 158 can be grooves that circumferentially extend around the second elongate shaft 150 and/or textured bands. In some embodiments, the gradation indicators 158 can be circumferential bands that circumferentially extend around the second elongate shaft 150. The circumferential bands can be formed from another type of material that is visually distinct from adjacent portions of the second elongate shaft 150. In some embodiments, the circumferential bands can be formed via paint or another type of colored and/or textured material that is applied to an exterior of the second elongate shaft 150.

In some embodiments, a distance at which the second elongate shaft 150 is protracted can be fixed through the inclusion of a locking device, disposed at an interface between the first elongate shaft 142 and the second elongate shaft 150. As depicted, the locking device can comprise a collet 160. The collet 160 can be configured to prevent longitudinal movement between the first elongate shaft 142 and the second elongate shaft 150. As discussed, the collet 160 can be disposed at an interface between the first elongate shaft 142 and the second elongate shaft 150. In some embodiments, the collet 160 can be disposed at a distal end of the first elongate shaft 142. For example, the collet 160 can be connected to a distal end of the first elongate shaft 142.

As depicted, in FIG. 2A, the collet 160 can include a barrel 162 and a collar 164. In some embodiments, the barrel can extend along the shaft longitudinal axis and can define a barrel lumen 166 that extends therethrough. The barrel lumen 166 can have an inner diameter that is the same or larger than an outer diameter of the second elongate shaft 150. In some embodiments, the collar 164 can extend along the longitudinal axis and can define a collar lumen 168 that extends therethrough. The collar lumen 168 can have an inner diameter that is the same or larger than an outer diameter of the barrel 162. In some embodiments, an exterior surface of the barrel 162 can be threaded and an interior surface of the collar 164 can be threaded, as further discussed in relation to FIG. 2B.

The barrel 162 can include a proximally extending mounting portion 170. The proximally extending mounting portion 170 can be a hollow cylindrical projection that proximally extends from a proximal end of the barrel 162. An inner diameter of the mounting portion 170 can be equal to the inner diameter of the barrel 162 and an outer diameter of the mounting portion 170 can be less than the outer diameter of the barrel 162. In some embodiments, the mounting portion 170 can be inserted into a recessed area of the first elongate shaft 142. For example, a distal face of the first elongate shaft 142 can include a recessed area sized and configured to accept the mounting portion 170. In some embodiments, the proximal end of the barrel 162 can be connected to a distal end of the first elongate shaft 142. In an example, the proximal end of the barrel 162 can be connected with the distal end of the first elongate shaft 142 with an adhesive and/or a mechanical fastener.

FIG. 2B depicts a cross-sectional side view of the collet 160 depicted in FIG. 2A along line 2B-2B, in accordance with embodiments of the present disclosure. The collet 160 can include the barrel 162 and collar 164. The barrel can define the barrel lumen 166 that extends therethrough. In some embodiments, the collar 164 can define the collar lumen 168 in which the barrel 162 can be disposed. The collar lumen 168 can have an inner diameter that is the same or larger than an outer diameter of the barrel 162. In some embodiments, an exterior surface of the barrel 162 can include barrel threads 172 and an interior surface of the collar 164 can include collar threads 174.

In some embodiments, an outer diameter of the barrel 162 can be tapered from a proximal end 176 to a distal end 178. For example, the outer diameter of the barrel 162 can increase from the proximal end 176 to the distal end 178. Accordingly, as the collar 164 is rotated and threaded along the barrel 162 proximally or distally, a diameter of the barrel lumen 166 at the distal end of the barrel can be increased and/or decreased. As the collar is threaded distally (e.g., moved to the right of the page), the collar can maintain a same diameter. Thus, the barrel 162 can be radially compressed, reducing the diameter of the barrel lumen 166 at the distal end of the distal end 178. In some embodiments, one or more compression slits 180 can be defined through a sidewall of the barrel 162. In an example, the compression slit 180 can extend proximally and longitudinally along the sidewall of the barrel 162 from the distal end 178. As the barrel 162 is radially compressed at the distal end, the compression slit 180 can allow for the distal end of the barrel 162 to be radially compressed inward toward a longitudinal axis of the barrel 162. Reduction in the diameter of the barrel lumen 166 can cause the inner walls of the barrel 162 to contact the second elongate shaft 150, which extends through the barrel lumen 166. In an example, a force can be applied to an outer wall of the second elongate shaft 150 via the inner walls of the barrel 162, causing the second elongate shaft 150 to be longitudinally held in place by the barrel 162.

With further reference to FIG. 2A, in some embodiments, an outer surface of the collar 164 can be textured (e.g., knurled) to improve a physician's grip on the collar 164 to enable the physician to rotate the collar 164. In some embodiments, the physician can rotate the collar 164 to distally advance the collar 164, causing the second elongate shaft 150 to be held in place.

FIG. 2C is a schematic and isometric side, top, and proximal end view of the adjustable catheter straightener 140 in FIG. 2A in an extended state, in accordance with embodiments of the present disclosure. The adjustable catheter straightener 140 depicted in FIG. 2C includes the same features as those discussed in FIGS. 2A and 2B, with the exception that the second elongate shaft 150 has been protracted with respect to the first elongate shaft 142. In an example, the seal 182 can be distally positioned within the first shaft lumen 148, such that the seal 182 abuts a proximal end of the barrel 162 (e.g., proximally extending mounting portion 170). In some embodiments, the seal 182 can serve as a stop portion, such that distal movement of the second elongate shaft 150 is limited when the seal 182 abuts the proximal end of the barrel 162. In an example, the first elongate shaft 142 can slide in relation to the seal 182 and the second elongate shaft 150, until the seal 182 abuts the proximal end of the barrel 162.

In some embodiments, the collet 160 can hold the second elongate shaft 150 in fixed longitudinal relation to the first elongate shaft 142. In an example, the collet 160 can be loosened and the second elongate shaft 150 can be longitudinally slid with respect to the first elongate shaft 142. When the second elongate shaft 150 is positioned in a desired position by a physician, the collet 160 can be tightened, thus locking the second elongate shaft 150 and the first elongate shaft 142 in a fixed relation to one another.

Although the seal 182 is depicted as being connected to the second shaft proximal end; in some embodiments, a seal can be disposed at a distal end of the first elongate shaft 142. Accordingly, the second elongate shaft 142 can be configured to slide in relation to a seal disposed at the distal end of the first elongate shaft 142.

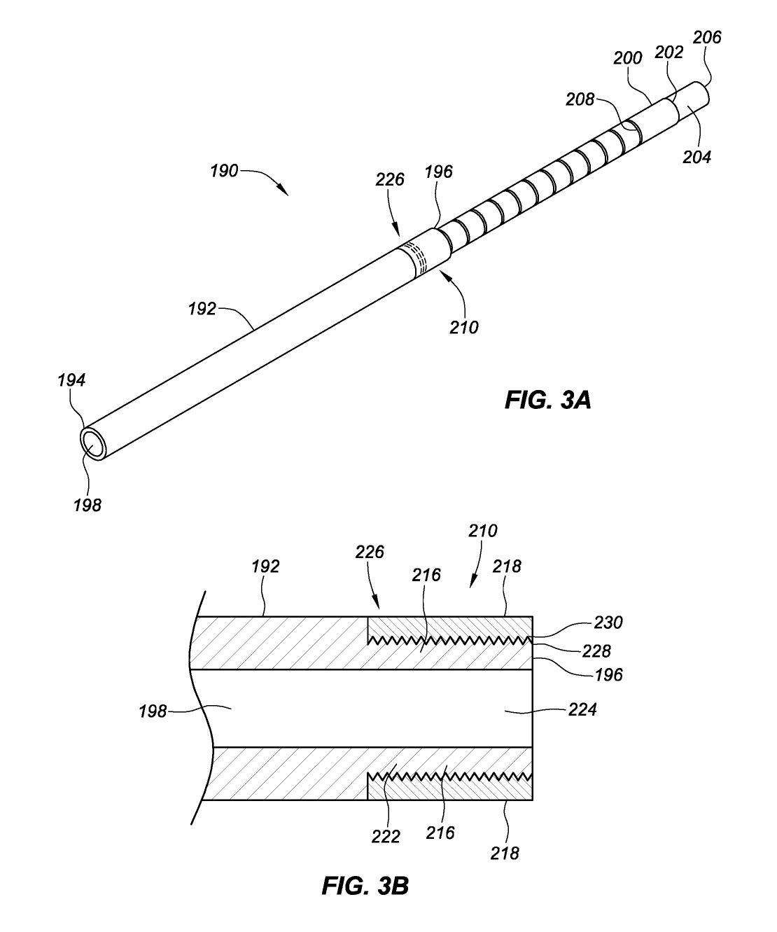

FIG. 3A is a side, top, and proximal end view of the adjustable catheter straightener 190 in an extended state, in accordance with embodiments of the present disclosure. As discussed herein, the adjustable catheter straightener 190 can include a first elongate shaft 192 that extends along a shaft longitudinal axis. The first elongate shaft 192 can include a first shaft proximal end 194 and a first shaft distal end 196. In some embodiments, the first elongate shaft 192 can include a first shaft inner wall that defines a first shaft lumen 198 that extends therethrough. In some embodiments, the catheter straightener 190 can include a second elongate shaft 200 that is disposed within the first shaft lumen 198 and that extends along the shaft longitudinal axis and comprises a second shaft proximal end (not shown) and a second shaft distal end 202. In some embodiments, a second shaft inner wall of the second elongate shaft 200 can define a second shaft lumen (not depicted) that extends therethrough. A flexible tip 204 can be connected to a second shaft distal end 202 of the second elongate shaft 200. In some embodiments, the first shaft lumen 198, the second shaft lumen, and a lumen formed by the flexible tip 204 can form a continuous lumen that extends from the first shaft proximal end 194 to a tip distal end 206. A catheter can be inserted into the first shaft lumen 198 and advanced through the catheter straightener 190, until the catheter is advanced from the tip distal end 206.

As further depicted, the catheter straightener includes one or more gradation indicators (e.g., gradation indicator 208). As previously discussed, the gradation indicators can indicate a length at which the second elongate shaft 200 has been retracted or protracted with respect to the first elongate shaft 192.

In some embodiments, the first elongate shaft 192 can include a collet 210 disposed at a distal end of the first elongate shaft 192. FIG. 3B depicts a cross-sectional view of the collet 210 and the first shaft distal end 196 in FIG. 3A, in accordance with embodiments of the present disclosure. In some embodiments, the collet 210 can be configured to prevent longitudinal movement between the first elongate shaft 192 and the second elongate shaft 200. As discussed, the collet 210 can be disposed at an interface between the first elongate shaft 192 and the second elongate shaft 200. In some embodiments, the collet 210 can be disposed at a distal end of the first elongate shaft 192. For example, the collet 210 can be connected to a distal end of the first elongate shaft 192.

As depicted, in FIG. 3B, the collet 210 can include a barrel 216 and a collar 218. In some embodiments, the barrel 216 can be formed from the first shaft distal end 196 of the first elongate shaft 192. In an example, the first shaft distal end 196 can have a reduced outer diameter, as depicted. For instance, a defined length of a distal most portion of the first elongate shaft 192 can have a reduced outer diameter with respect to proximal portions of the first elongate shaft 192. The barrel can extend along a shaft longitudinal axis of the catheter straightener 190 and can define a barrel lumen 224, which is collinear with the first shaft lumen 198. In some embodiments, the collar 218 can extend along the longitudinal axis and can define a collar lumen 222 that extends therethrough. The collar lumen 222 can have an inner diameter that is the same or larger than an outer diameter of the barrel 216. In some embodiments, an exterior surface of the barrel 216 can include barrel threads 228 and an interior surface of the collar 218 can include collar threads 230.

In some embodiments, an inner diameter of the collar 218 can be tapered as it extends distally. For example, the inner diameter of the collar 218 can be reduced in a distal direction. Thus, as the collar 218 is proximally threaded onto the barrel 216, the barrel 216 can be compressed by the collar 218 and a diameter associated with the barrel lumen 224 can be reduced. Accordingly, the second elongate shaft 200 can be clamped in place by an inner wall of the barrel lumen 224. In some embodiments, the barrel can include a compression slit, as discussed in relation to FIG. 2B. As depicted in FIGS. 3A and 3B, an outer surface of the collar 218 can be flush with an outer surface of the first elongate shaft 192. In some embodiments, the collar 218 can include one or more texture surfaces 226 to provide a physician with an improved grip.

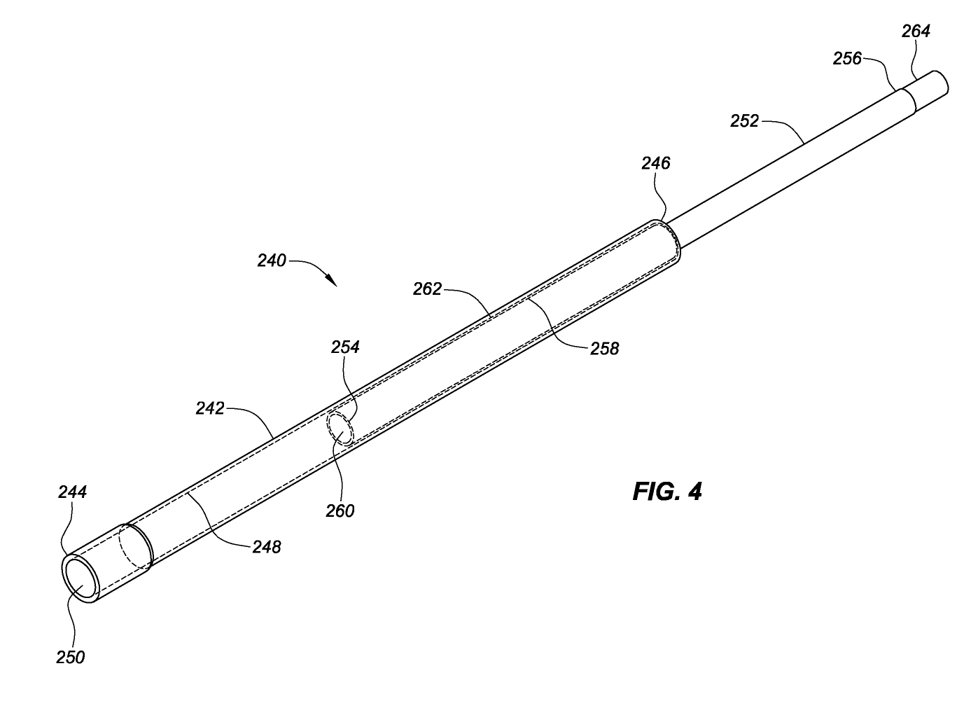

FIG. 4 is a schematic and isometric side, top, and proximal end view of an adjustable catheter straightener 240 that includes a static locking device, in accordance with embodiments of the present disclosure. The catheter straightener 240 can include a first elongate shaft 242 that extends along a shaft longitudinal axis. The first elongate shaft 242 can include a first shaft proximal end 244 and a first shaft distal end 246. In some embodiments, the first elongate shaft 242 can include a first shaft inner wall 248 that defines a first shaft lumen 250 that extends therethrough. In some embodiments, the catheter straightener 240 can include a second elongate shaft 252 that is disposed within the first shaft lumen 250 and that extends along the shaft longitudinal axis and comprises a second shaft proximal end 254 and a second shaft distal end 256. In some embodiments, a second shaft inner wall 258 of the second elongate shaft 252 can define a second shaft lumen 260 that extends therethrough. In some embodiments, the first shaft lumen 250 and the second shaft lumen 260 can form a continuous lumen that extends from the first shaft proximal end 244 to the second shaft distal end 256. As discussed herein, in some embodiments, a flexible tip 264 can be disposed at the second shaft distal end 256. The flexible tip 264 can define a tip lumen (not depicted) that can form a continuous lumen with the first shaft lumen 250 and the second shaft lumen 260. A proximal end of the first elongate shaft 242 can define an expanded lumen, which can aid in insertion of a catheter into the catheter straightener 240.

In some embodiments, as discussed herein, the catheter straightener 240 can include a locking device that is configured to prevent longitudinal movement between the first elongate shaft 242 and the second elongate shaft 252. In some embodiments, the locking device can be a static locking device. In an example, the static locking device 128 can include a frictional fit between the first elongate shaft 242 and the second elongate shaft 252. For instance, the outer diameter of the second elongate shaft 252 can be the same or slightly larger than the inner diameter of the first elongate shaft 242, which can create friction between the first shaft inner wall 248 and an outer wall of the second elongate shaft 252. In some embodiments, a distal inner diameter of the first elongate shaft 242 can be gradually decreased (e.g., tapered) and a proximal outer diameter of the second elongate shaft 252 can be gradually increased. Accordingly, as the second elongate shaft 252 is protracted with respect to the first elongate shaft 242, a frictional fit can exist between a distal portion of the first shaft inner wall 248 and a proximal portion of a second shaft outer wall 262.

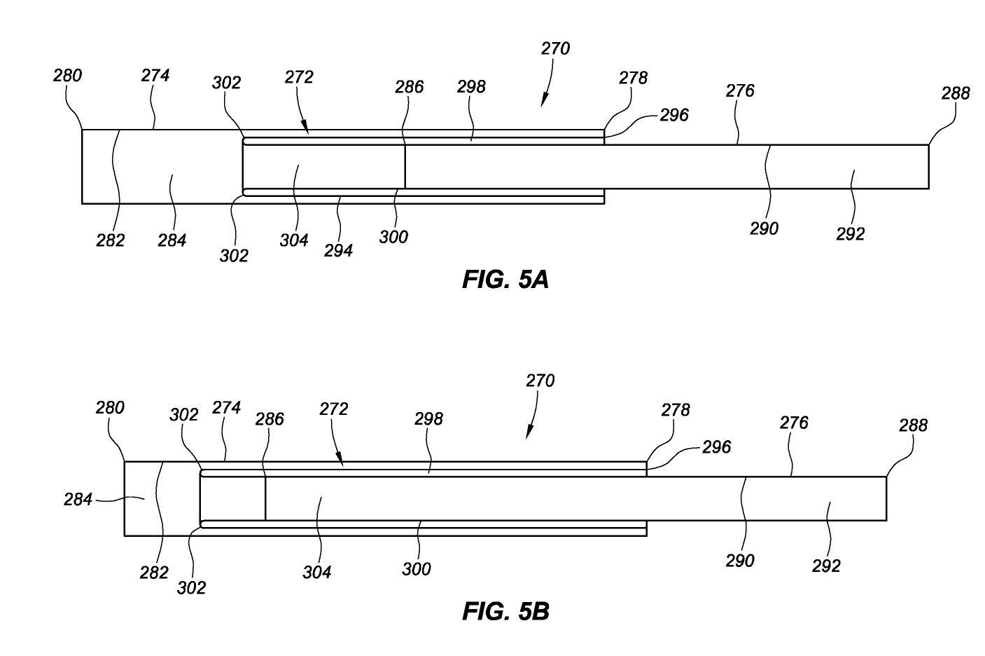

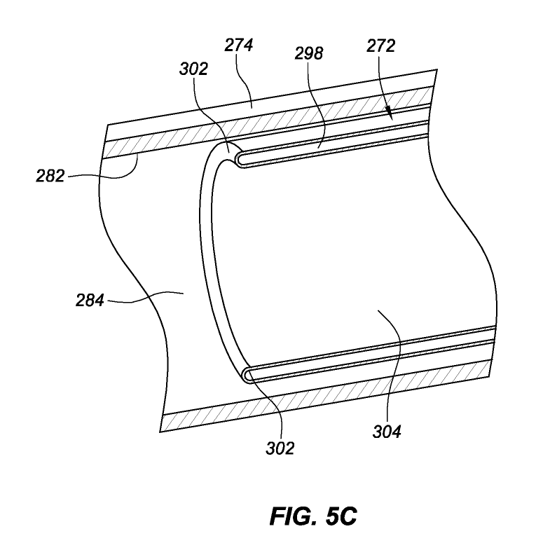

FIG. 5A depicts a cross-sectional side view of an adjustable catheter straightener 270 in a partially extended state with an interface seal 272 disposed between a first elongate shaft 274 and a second elongate shaft 276, in accordance with embodiments of the present disclosure. The catheter straightener 270 can include the first elongate shaft 274 that extends along a shaft longitudinal axis. The first elongate shaft 274 can include a first shaft proximal end 280 and a first shaft distal end 278. In some embodiments, the first elongate shaft 274 can include a first shaft inner wall 282 that defines a first shaft lumen 284 that extends therethrough. In some embodiments, the catheter straightener 270 can include a second elongate shaft 276 that is disposed within the first shaft lumen 284 and that extends along the shaft longitudinal axis and comprises a second shaft proximal end 286 and a second shaft distal end 288. In some embodiments, a second shaft inner wall 290 of the second elongate shaft 276 can define a second shaft lumen 292 that extends therethrough. In some embodiments, the first shaft lumen 284 and the second shaft lumen 292 can form a continuous lumen that extends from the first shaft proximal end 280 to the second shaft distal end 288. As discussed herein, in some embodiments, a flexible tip can be disposed at the second shaft distal end 288, although not depicted.

In some embodiments, an interface seal 272 can be formed between the distal end of the first shaft distal end 278 and the second shaft proximal end 286. As depicted, an interface distal end 296 of a flexible interface 294, also referred to herein as flexible interface 294, can be attached to the first shaft inner wall 282 and/or first shaft distal end 278. The flexible interface 294 can extend proximally from the first shaft distal end 278 through the first shaft lumen 284 forming an interface lumen 298 and can be a flexible hollow cylindrical piece of material. The proximal most portion of the flexible interface seal 272 can be folded back into the interface lumen 298 and an interface proximal end 300 can be attached to the second shaft proximal end 286. A proximal most portion of the flexible interface seal 272 can include a transition point 302, where the flexible interface seal 272 folds upon itself

In some embodiments, the second elongate shaft 276 can be longitudinally extended or retracted with respect to the first elongate shaft 274. When the second elongate shaft 276 is moved proximally with respect to the first elongate shaft 274, the transition point can be moved proximally, maintaining a seal between the first shaft distal end 278 and the second shaft proximal end 286. Alternatively, when the second shaft is moved distally with respect to the first elongate shaft 274, the transition point can be moved distally, maintaining a seal between the first shaft distal end 278 and the second shaft proximal end 286.

For instance, FIG. 5B depicts a cross-sectional side view of the adjustable catheter straightener 270 in FIG. 5A in a partially retracted state, in accordance with embodiments of the present disclosure. As depicted, the second shaft has been moved proximally with respect to the first elongate shaft 274. Accordingly, the interface proximal end 300 and the transition point 302 have been moved proximally. In some embodiments, the interface seal 272 can define a central interface lumen 304, which together with the first shaft lumen 284 and the second shaft lumen 292 form a continuous lumen through which a catheter can be disposed.

FIG. 5C is a cross-sectional side, top, and proximal end view of the transition point 302 associated with the interface seal 272 and the first elongate shaft 274 depicted in FIG. 5A, in accordance with embodiments of the present disclosure. As depicted, the interface seal extends proximally along the first shaft inner wall 282, before being folded back into the interface lumen 298 at the transition point 302. Although not shown, the distal end of the interface distal end can be attached to the distal end of the first elongate shaft 274 and the interface proximal end can be attached the proximal end of the second elongate shaft 276, forming an interface seal 272 between the two shafts. The first shaft lumen 284, the central interface lumen 304, and the second shaft lumen can together form a continuous lumen in which a catheter can be disposed.

Reference throughout the specification to “various embodiments,” “some embodiments,” “one embodiment,” or “an embodiment”, or the like, means that a particular feature, structure, or characteristic described in connection with the embodiment(s) is included in at least one embodiment. Thus, appearances of the phrases “in various embodiments,” “in some embodiments,” “in one embodiment,” or “in an embodiment,” or the like, in places throughout the specification, are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features, structures, or characteristics of one or more other embodiments without limitation given that such combination is not illogical or non-functional.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

Although at least one embodiment of an adjustable catheter straightener has been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the devices. Joinder references (e.g., affixed, attached, coupled, connected, and the like) are to be construed broadly and can include intermediate members between a connection of elements and relative movement between elements. As such, joinder references do not necessarily infer that two elements are directly connected and in fixed relationship to each other. It is intended that all matter contained in the above description or shown in the accompanying drawings shall be interpreted as illustrative only and not limiting. Changes in detail or structure can be made without departing from the spirit of the disclosure as defined in the appended claims.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed:

1. An adjustable catheter straightener, comprising:
a first elongate shaft extending along a shaft longitudinal axis and comprising a first shaft proximal end and a first shaft distal end, wherein a first shaft inner wall of the first elongate shaft defines a first shaft lumen extending therethrough;
a second elongate shaft disposed within the first shaft lumen extending along the shaft longitudinal axis and comprising a second shaft proximal end and a second shaft distal end, wherein:
a second shaft inner wall of the second elongate shaft defines a second shaft lumen extending therethrough, the first shaft lumen and the second shaft lumen forming a continuous lumen; and
the first elongate shaft and the second elongate shaft are configured to move longitudinally with respect to one another.

2. The adjustable catheter straightener of claim 1, wherein:
the second elongate shaft includes a second shaft outer wall; and
a seal is disposed between the first shaft inner wall and the second shaft outer wall.

3. The adjustable catheter straightener of claim 2, wherein the seal is connected to the second shaft proximal end.

4. The adjustable catheter straightener of claim 3, wherein the first elongate shaft is configured to slide with respect to the seal.

5. The adjustable catheter straightener of claim 2, wherein the seal is connected to the first shaft distal end.

6. The adjustable catheter straightener of claim 5, wherein the second shaft is configured to slide with respect to the seal.

7. The adjustable catheter straightener of claim 1, wherein:
the second shaft distal end includes a flexible tip; and
the second elongate shaft is formed from a material with a flexibility that is less than the flexible tip.

8. The adjustable catheter straightener of claim 7, wherein the flexible tip is formed from a material with a durometer in a range from 25D to 55D.

9. The adjustable catheter straightener of claim 7, wherein the flexible tip extends distally from the second shaft distal end.

10. The adjustable catheter straightener of claim 1, further comprising a locking device configured to prevent longitudinal movement between the first elongate shaft and the second elongate shaft.

11. The adjustable catheter straightener of claim 10, wherein the locking device is disposed at an interface between the first elongate shaft and the second elongate shaft.

12. An adjustable catheter straightener, comprising:
a first elongate shaft extending along a shaft longitudinal axis and comprising a first shaft proximal end and a first shaft distal end, wherein a first shaft inner wall of the first elongate shaft defines a first shaft lumen extending therethrough;
a second elongate shaft disposed within the first shaft lumen extending along the shaft longitudinal axis and comprising a second shaft proximal end and a second shaft distal end, wherein a second shaft inner wall of the second elongate shaft defines a second shaft lumen extending therethrough; and
a flexible tip disposed at the second shaft distal end, wherein a tip inner wall of the flexible tip defines a tip lumen, wherein the first shaft lumen, the second shaft lumen, and the tip lumen form a continuous lumen.

13. The adjustable catheter straightener of claim 12, wherein a locking device is disposed at the first shaft distal end.

14. The adjustable catheter straightener of claim 12, wherein a proximal end of the first shaft inner wall is tapered radially outward with respect to the shaft longitudinal axis.

15. The adjustable catheter straightener of claim 12, wherein a seal is disposed around an outer circumference of the second shaft proximal end.

16. The adjustable catheter straightener of claim 12, wherein a plurality of gradation indicators are disposed on a portion of a longitudinal length of the second elongate shaft.

17. The adjustable catheter straightener of claim 16, wherein each of the gradation indicators circumferentially extends around the second elongate shaft.

18. An adjustable catheter straightener, comprising:
a first elongate shaft extending along a shaft longitudinal axis and comprising a first shaft proximal end and a first shaft distal end, wherein a first shaft inner wall of the first elongate shaft defines a first shaft lumen extending therethrough;
a second elongate shaft disposed within the first shaft lumen extending along the shaft longitudinal axis and comprising a second shaft proximal end and a second shaft distal end, wherein a second shaft inner wall of the second elongate shaft defines a second shaft lumen extending therethrough;
a flexible tip disposed at the second shaft proximal end, wherein a tip inner wall of the flexible tip defines a tip lumen, wherein the first shaft lumen, the second shaft lumen, and the tip lumen form a continuous lumen; and
a locking device configured to prevent longitudinal movement between the first elongate shaft and the second elongate shaft.

19. The adjustable catheter straightener of claim 18, wherein the locking device comprises a collet.

20. The adjustable catheter straightener of claim 18, wherein the locking device comprises a static locking device.
